(19) United States
(12) Patent Application Publication
(10) Pub. No.: US 2010/0294280 A1
(43) Pub. Date: Nov. 25, 2010

(54) BREATHING TUBE SYSTEM COMPRISING A HEATING ELEMENT

(75) Inventor: Markus Kratzenstein, Lubeck (DE)

Correspondence Address: MCGLEW & TUTTLE, PC, P.O. BOX 9227, SCARBOROUGH STATION, SCARBOROUGH, NY, 10510-9227, US

(73) Assignee: DRÄGER MEDICAL AG & CO. KG, Lübeck (DE)

(21) Appl. No.: 12/445,810

(22) PCT Filed: Oct. 24, 2007
(86) PCT No.: PCT/DE07/01920

§ 371 Date: Apr. 16, 2009

(30) Foreign Application Priority Data
Nov. 10, 2006 (DE) 10 2006 052 997.9

(51) Int. Cl. A61M 16/10

(52) U.S. Cl. 128/204.17

(57) ABSTRACT

A breathing tube system has a heating element, wherein a resistance wire extends several times to and fro in the longitudinal direction of a tube with wire terminals at one end and is held in the tube in parallel by means of a plurality of spreading elements located at spaced locations from one another in the longitudinal direction of the tube. The resistance wire extends at least four times in the longitudinal direction of the tube. The spreading elements have guide elements for receiving the resistance wire at the inner tube wall of the tube. The spreading elements with the resistance wire are displaceable in the tube in the longitudinal direction.

### CROSS REFERENCE TO RELATED APPLICATIONS

This application is a U.S. National Phase application of International Application PCT/DE2007/001920 and claims the benefit of priority under 35 U.S.C. §119 of German Patent Application DE 10 2006 052 997.9 filed Nov. 10, 2009, the entire contents of which are incorporated herein by reference.

### FIELD OF THE INVENTION

The present invention pertains to a breathing tube system with a heating element.

### BACKGROUND OF THE INVENTION

There are various breathing tube systems in clinical practice for supplying a mechanically respirated patient, in which breathing gas is actively heated over the length of the tube in order to prevent an excessively intense cooling of the breathing gas at the tube wall or walls. This is usually achieved by one or more heating elements, which generate thermal output in one or more resistance wires by imposing an electrical current, being introduced into the tube. The generated heat is released to the breathing gas flowing through especially by convection.

In the prior-art systems with resistance wires of a defined resistance, these [resistance wires] are incorporated in the tube walls along the tube. A resistance wire is usually led from the respirator-side terminal to the patient-side terminal. The resistance wire is led out of the breathing tube system on the respirator side and is electrically contacted. The resistance wire is usually held on the patient side by a clamping element to ensure that it will remain uniformly distributed in the tube during the operation as well. However, said clamping element limits the freedom of motion of the tube in the longitudinal axial direction.

Due to the fact that the resistance wire is led forward and returned only once, the wire surface relevant for the release of heat is provided with a high temperature during the operation in order to release a defined thermal output to the breathing gas flowing through. Since the temperatures are often too high for the tube material being used, the resistance of the resistance wire must be selected to be such that the temperatures are limited, but this will have the undesired consequence that the required thermal power will not be released in the tube any longer.

This problem is solved in other breathing tube systems by the resistance wire in the tube being designed as a helix or as a double helix. A double helix is formed by the intrinsic stress of the resistance wire or of the insulation material. A relatively close approach to the tube wall is achieved and a multiple of the tube length can be introduced into the tube in these breathing tube systems. However, crossing points, at which two resistance wires cross each other and lie one on top of another and thus touch each other after each half revolution of the helix, develop in these breathing tube systems due to the course that is helical on both sides. Great temperature elevations can develop at these points due to the lack of heat dissipation at the contact points. This represents a risk of melting especially in case of tube materials with a low melting point and also implies an increased risk of inflammation of the tube material used.

Another drawback of the double helix is the development of highly turbulent flows in the breathing gas accompanied by a high flow resistance in the tube. Swirling develops in the breathing gas due to the wire extending at right angles to the flow of the breathing gas.

The breathing tube systems that are currently available commercially have the further drawback that the resistance wire or resistance wires of the helix or double helix design must be pulled into the tube with a tool, so that the tube may be easily damaged.

A breathing tube system designed as a breathing gas humidifier appears from U.S. Pat. No. 3,871,373, where water is led in the longitudinal direction to and fro in the breathing tube system proper in a tube releasing water vapor.

A breathing tube system with a heating element led centrally at a spaced location from the tube wall is described in DE 196 47 548 C2, wherein the heating line is held by means of at least one thermally insulated spacer.

### SUMMARY OF THE INVENTION

The object of the present invention is to provide an improved breathing tube system, which can be embodied in a simple manner, with a heating element, so that the most uniform possible release of heat to the breathing gas flowing through is possible at a relatively low temperature of the heating element.

According to the invention, a breathing tube system is provided with a heating element, wherein a resistance wire extends several times to and fro in the longitudinal direction of the tube. Wire terminals are provided at one end in a tube. The wires are held in parallel by means of a plurality of spreading elements located at spaced locations from one another in the longitudinal direction of the tube. The spreading elements have guide elements for receiving the resistance wire at an inner tube wall. The resistance wire extends at least four times in the longitudinal direction of the tube. The spreading elements with the resistance wire are displaceable in the tube in the longitudinal direction thereof.

At least three spreading elements may advantageously be arranged at spaced locations over the length of the tube. Each spreading element may advantageously have at least three or four guide elements for receiving the resistance wire.

The wire terminals may advantageously be arranged at the device-side end of the tube.

The resistance wire may advantageously comprise a plurality of strands, which are preferably coated together by means of an electrically insulating plastic, such as especially silicone.

The spreading elements may advantageously be provided with a plurality of spreading arms with a guide element each corresponding to the number of resistance wires to be received. The spreading elements may advantageously be arranged equidistantly, especially in the form of a cross.

The tube may have a length of about 1 m to 1.5 m and an internal diameter of about 10 mm to 30 mm.

The breathing tube system according to FIG. 1 has the essential advantage that a resistance wire length corresponding to a multiple of the length of the breathing tube is integrated in the tube and it is possible as a result to work at a low resistance wire temperature, so that no overheating effects can occur in the tube material and good heat transmission to the breathing gas flowing through is achieved. This is achieved ultimately by the resistance wires extending in the tube essentially in parallel to the longitudinal direction of the tube and being led forward and back several times and by contact crossings being extensively ruled out. The resistance wires are led for this along the length of the tube at spaced locations by means of a plurality of spreading elements, which are introduced into the tube especially with the resistance wires. The spreading elements are designed such that they lead the preferably one resistance wire laterally as close to the tube wall as possible, so that no water of condensation will form there due to the released heat. The resistance wire is prevented from collapsing in the tube and entanglement is consequently prevented by the selected spacing of the spreading elements in the longitudinal direction of the tube and by the intrinsic rigidity of the resistance wire.

An exemplary embodiment of the present invention will be explained below on the basis of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a section through a tube of a breathing tube system with a spreading element with four spreading arms for receiving the resistance wire; and

FIG. 2 is a view of a breathing tube system in the longitudinal section.

### DESCRIPTION OF THE PREFERRED EMBODIMENTS

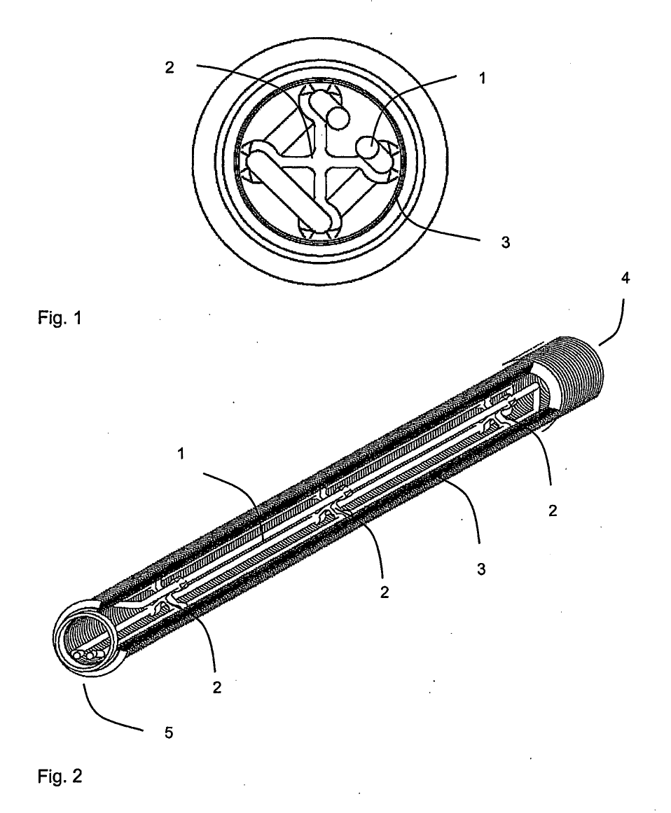

Referring to the drawings in particular, the spreading elements 2, which are supported in the tube wall 3 according to FIG. 1, have a plurality of, for example, four spreading arms, which are as narrow as possible, for receiving and guiding a resistance wire 1 possibly at the tube wall 3 and fit into the inner contour of the tube. The number of guide elements depends on the number of courses of resistance wire 1 in the longitudinal direction of the tube.

In the embodiment shown in FIGS. 1 and 2, the resistance wire 1 extends twice from the anesthesia apparatus or respirator or from the device-side end 5 to the patient-side end 4 and back. The electrically conductive part of the resistance wire 1 consists especially of a plurality of strands in order to prevent elevations of resistance in case of a cable break. The strands are preferably coated with a common outer insulating layer consisting of temperature-resistant silicone or another suitable material such as polypropylene.

The resistance wire 1 is held by, for example, three spreading elements 2 arranged at spaced locations from one another along the longitudinal direction of the tube, one spreading element being located at or in front of the device-side end 5, one at or in front of the patient-side end 4 and one approximately in the middle of the tube at the tube wall 3. The resistance wire 1 is placed, for example, fourfold into the tube and clamped in the spreading elements 2, so that the resistance wire 1 is led to and fro twice when viewed from the device. Both electrical contacts are led out of the tube at the device-side end 5 and contacted in the anesthesia apparatus or respirator, not shown in more detail. The length of the breathing tube system is approx. 1 m to 1.5 m, and the diameter is about 10 mm to 30 mm depending on the particular application.

The following can be mentioned as essential advantages of the present breathing tube system:

By inserting the resistance wire 1 fourfold or more over the length of the tube and the essentially parallel guiding at the inner tube wall 3 in the longitudinal direction of the tube, improved distribution of heat dissipation to the breathing gas flowing through is achieved. In addition, it is achieved by the parallel guiding of the resistance wires 1 in the tube that the flow resistance in the tube system remains acceptably low, and a nearly laminar flow is achieved by the parallel positioning of the spreading elements 2 in the tube. At the same time, the resistance wires 1 are led in an essentially contactless manner. In addition, the resistance wire 1 with the guiding spreading elements 2 can be inserted into the tube manually without problems.

While specific embodiments of the invention have been shown and described in detail to illustrate the application of the principles of the invention, it will be understood that the invention may be embodied otherwise without departing from such principles.

1-9. (canceled)

10. A breathing tube system comprising:
a breathing tube with an inner tube wall;
a heating element with a resistance wire extending several times to and fro in the longitudinal direction of said tube and with wire terminals at one end;
a plurality of spreading elements spaced apart from each other in the longitudinal direction of the tube, said resistance wire being held in parallel in the tube by said spreading elements with said resistance wire extending at least four times in the longitudinal direction of the tube, said spreading elements having guide elements for receiving the resistance wire at said inner tube wall, said spreading elements being displaceable with the resistance wire in the longitudinal direction of said tube.

11. A breathing tube system in accordance with claim 10, wherein:
said plurality of spreading elements comprise at least three spreading elements arranged at spaced locations from one another over the length of said tube; and
each of said spreading elements has at least four guide elements for receiving said resistance wire.

12. A breathing tube system in accordance with claim 10, wherein said wire terminals are arranged at the device-side end of the tube.

13. A breathing tube system in accordance with claim 10, wherein said resistance wire comprises a plurality of strands, said strands being coated together by an electrically insulating plastic.

14. A breathing tube system in accordance with claim 13, wherein said electrically insulating plastic is a silicone material.

15. A breathing tube system in accordance with claim 10, wherein said spreading elements are each provided with a plurality of spreading arms with a guide element corresponding to a number of portions of said resistance wire to be received.

16. A breathing tube system in accordance with claim 15, wherein said spreading elements are arranged equidistantly.

17. A breathing tube system in accordance with claim 16, wherein said spreading elements are arranged in the form of a cross.

18. A breathing tube system in accordance with claim 10, wherein said tube has a length from 1 m to 1.5 m and an internal diameter from about 10 mm to 30 mm.

19. A breathing tube system comprising:
a breathing tube with an inner tube wall;
a heating element with a resistance wire with four longitudinal direction extensions of a length of said tube and with wire terminals at one end;
a plurality of spreading elements spaced apart from each other in the longitudinal direction of the tube, each of said extensions of said resistance wire being held in parallel in the tube by said spreading elements, said spreading elements having guide elements, each of said guide elements for receiving one of said extensions of said resistance wire at said inner tube wall, said spreading elements being displaceable with the resistance wire in the longitudinal direction of said tube.

20. A breathing tube system in accordance with claim 19, wherein:
said plurality of spreading elements comprise at least three spreading elements arranged at spaced locations from one another over the length of said tube; and
each of said spreading elements has at least said four guide elements for receiving a respective one of said four extensions of said resistance wire.

21. A breathing tube system in accordance with claim 19, wherein said wire terminals are arranged at a device-side end of the tube.

22. A breathing tube system in accordance with claim 19, wherein said resistance wire comprises a plurality of strands, said strands being coated together by an electrically insulating plastic.

23. A breathing tube system in accordance with claim 22, wherein said electrically insulating plastic is a silicone material.

24. A breathing tube system in accordance with claim 19, wherein said spreading elements are each provided with a plurality of spreading arms with a guide element corresponding to a number of resistance wire extensions to be received.

25. A breathing tube system in accordance with claim 24, wherein said spreading elements are arranged equidistantly.

26. A breathing tube system in accordance with claim 25, wherein said spreading elements are arranged in the form of a cross.

27. A breathing tube system in accordance with claim 19, wherein said tube has a length from 1 m to 1.5 m and an internal diameter from about 10 mm to 30 mm.
